(19) United States
(12) Patent Application Publication
(10) Pub. No.: US 2009/0042983 A1
(43) Pub. Date: Feb. 12, 2009

(54) Use of L-carnitine for the treatment of cardiovascular diseases

(75) Inventor: Aleardo Koverech, Rome (IT)

Correspondence Address: NIXON & VANDERHYE, PC, 901 NORTH GLEBE ROAD, 11TH FLOOR, ARLINGTON, VA, 22203, US

(73) Assignee: Sigma-Tau Industrie Farmaceutiche Riunite S.p.A., Rome (IT)

(21) Appl. No.: 12/232,321

(22) Filed: Sep. 15, 2008

Related U.S. Application Data
(63) Continuation-in-part of application No. 10/538,868, filed on Jun. 14, 2005.

(30) Foreign Application Priority Data
Apr. 17, 2003 (IT) RM2003A000178

(51) Int. Cl. A61K 31/205

(52) U.S. Cl. 514/554

(57) ABSTRACT

The use of L-carnitine or one of its pharmaceutically acceptable salts is described for the preparation of a medicine useful for reducing the number of deaths caused by acute myocardial infarction and for improving the short-and-long-term prognosis in the patients treated with it, in which L-carnitine is administered parenterally within the first few hours of onset of the symptoms of acute myocardial infarction at an initial dose of 9 grams a day for 5 days, after which the treatment is continued at a dose of 4 grams a day by the enteral route

This application is a continuation-in-part of application Ser. No. 10/538,868 filed Jun. 14, 2005, which in turn is a U.S. national phase of international application PCT/IT2004/000107, filed 3 Mar. 2004, which designated the U.S. and claims priority of IT RM2003 A 000178, dated 17 Apr. 2003, the entire contents of each of which are hereby incorporated by reference.

The invention described herein relates to the use of L-carnitine as a medicine useful for reducing the number of deaths caused by acute myocardial infarction and for improving the short- and long-term prognosis in the patients treated, in which the L-carnitine is administered parenterally within the first few hours of onset of the symptoms of acute myocardial infarction, at an initial dose of 9 grams a day for 5 days, after which the treatment is continued at a dose of 4 grams a day by the enteral route.

Acute myocardial infarction (AMI) causes morphofunctional alterations that often induce progressive left ventricular dilatation (“ventricular remodelling” phenomenon).

Post-AMI ventricular dilatation can be regarded as an overall compensation mechanism aimed at maintaining an adequate cardiac output in the presence of a reduction of the ejection fraction.

The extent of the ventricular dilatation is the most important prognostic indicator in patients with AMI.

Patients with relatively larger ventricular volumes are at greater risk of future cardiac events (Circulation 1987; 76:44-51).

Limiting the ventricular remodelling phenomenon in the post-infarction period is thus of great importance from the clinico-prognostic point of view (Circulation 1994; 89:68-75). Limitation of this phenomenon can be achieved by two mechanisms: (a) by limiting the extent of the infarcted area (which is the main determinant of future dilatation) by means of early myocardial reperfusion (Circulation 1989; 79:441-444) and/or (b) by reducing the parietal stress and consequently the progressive dilatation of the myocardial area not involved in the infarction process by means of the administration of ACE inhibitors.

When the thrombotic obstruction evolves rapidly towards complete, permanent, vascular occlusion, the resulting lack of perfusion gives rise, in the space of a few hours, to myocardial cell necrosis and thus to infarction. The immediate and long-term prognosis will depend upon a series of factors, the most important of which are the size of the necrotic area and the early and late complications resulting from it. It is therefore obvious that the primary aim of modern therapy for acute infarction is to reduce the size of the infarcted area. This objective is achieved with reperfusion procedures, whether pharmacological (thrombolytic agents), mechanical (PTCA), such as angioplasty, or surgical (by-pass). Generally, the earlier and more effective the reperfusion, the smaller will be the necrotic area. The latter is also influenced, albeit to a lesser extent, by other factors, and above all by the consumption of myocardial oxygen, which is conditioned by the object's heart rate, myocardial contractility and parietal tension. Of fundamental importance, then, will be all those measures, whether pharmacological or otherwise, that reduce cardiac work, while at the same time maintaining an adequate circulatory capacity.

More than half of all subjects that die of infarction do so during the first few hours.

Useful drugs for the treatment of acute myocardial infarction are already known.

Beta-blockers are drugs endowed with antiarrhythmia properties and are significantly more active if used in the early stages of the onset of the infarction.

Nitroderivatives are drugs administered usually by venous infusion and are useful for enhancing myocardial perfusion through the vasodilatation of the epicardial vessels.

Sodium nitroprussiate is a drug that exerts a double action on the arteriolar and venous districts. This compound produces coronary and renal vasodilatation, thus enhancing myocardial perfusion and diuresis.

L-carnitine is a known compound, the preparation procedure of which is described in U.S. Pat. No. 4,254,053.

The use of L-carnitine for the treatment of cardiac diseases is already well known.

In Drugs Exp Clin Res 1992; 18(8):355-65, the authors describe the use of L-carnitine in infarct victims, in which oral treatment with L-carnitine was initiated after the patients had been discharged from hospital. In this report, the authors do not describe or suggest that L-carnitine is useful in preventing death in the course of acute myocardial infarction.

In Eur Heart J 1989 June; 10(6):502-8, the authors describe the use of L-carnitine in infarct victims, in which the antiarrhythmia and metabolic effects of L-carnitine are evaluated. In this study it is reported that there were two deaths each in the group treated with L-carnitine and in that treated with placebo, respectively.

In J Am Coll Cardiol 1995 August; 26(2):380-7, the authors investigate the prolonged use of L-carnitine in patients affected by anterior acute myocardial infarction, and its effect on left ventricular volume at 3, 6 and 12 months after the emergent event. In this study L-carnitine was administered within 24 hours from the onset of the chest pain and the results given start from 3 months up to 1 year.

Incidentally deaths were recorded: 11 patients in the treated group and 14 in the control group died during the hospitalisation period. The Authors conclude that this slight reduction of deaths in short- and long-term is not significant due to the small size of the set of patients (472 in total). Moreover, the same Authors admit that their study is unsuitable for assessing survival rate and to find a way to reduce deaths in particular in the short-term. Moreover, they underline that their study was purposively limited to a specific subpopulation of patients, namely only those affected by anterior acute myocardial infarction and that the results cannot be extrapolated to patients with other acute myocardial infarction lesions.

Therefore, the administration of L-carnitine in this specific subpopulation of patients suffering from anterior acute myocardial infarction led to a significant attenuation of left ventricular dilation in the first year after occurrence of infarction, thus possibly reducing the future risk of cardiac failure. However, no beneficial effects on survival after infarction was given. Moreover, the Authors admitted their result may not be extended to the general population of subject suffering from infarct.

U.S. patent application, published with the No. US20020002202, discloses the use of fumarate salt of C₂-C₈ alkanoyl carnitines, for the prevention of organ ischemia. In particular, the examples refer to the effect of L-carnitine Fumarate on perfused heart according to the model disclosed by Bolukoglu H., et al., Am. J. Physiol:, 1996: 270; H817-26. Cardiac functions, hemodynamics, metabolites and enzymes were monitored. The results show that the hearts treated with L-carnitine fumarate have reduced release of LDH, higher reserve of high-energy phosphate, lower lactate levels. Regarding haemodynamic functions, the postischemic cardiac contraction amplitude and the coronary flow were greater in the hearts treated with carnitine fumarate.

The teaching of the above US 20020002202 is limited to isolated heart and could not be extended to a clinical forecast on living subjects, especially in estimating possible increase in survival after infarction.

In Am Heart J 2000 February; 139(2 Pt 3):S115-9, which is a review of the metabolic effects of L-carnitine in the cardiological field, the authors report that L-camitine is effective because it has metabolic effects on lipid and glucose metabolism.

In Lancet 1982 Jun. 19; 1(8286):1419-20, the authors report that analyses of cardiac tissue samples from patients who died of infarction, in parallel with samples of cardiac tissue from subjects who died of diseases other than infarction, show that, in the cardiac areas not affected by infarction (of the heart disease patients) the levels of free carnitine were the same as those in controls, whereas the free carnitine levels in areas of infracted cardiac tissue were lower than in controls.

In Postgrad Med J 1996 January; 72(843):45-50, the authors describe the use of L-carnitine in patients manifesting infarction symptoms in the 24-hour period prior to the start of treatment. In this study, L-carnitine was administered at a dose of 2 g/day, and the number of deaths at 28 days after the start of treatment was 6 in the control group and 4 in the treated group. The non-significance of the difference in the number of deaths recorded in the two groups tested is evident.

In Am J Cardiovasc Pathol 1990; 3(2):131-42, the authors describe the use of L-carnitine in an experimental model of cardiac ischaemia in experimental animals (dogs), in which L-carnitine proved to be active in improving cardiac lipid metabolism in these animals.

There are numerous other publications dealing with the use of L-carnitine in the cardiological field; neither these nor the above-mentioned publications describe or suggest the use of L-carnitine as a medicine useful for reducing the number of deaths caused by acute myocardial infarction, in which L-carnitine is administered intravenously within the first few hours of onset of acute myocardial infarction symptoms.

The only document of known technique to be found which described the use of L-carnitine within the first few hours of acute myocardial infarction is in Drugs Exptl. Clin. Res. X(4) 219-223 (1984). In this publication, the authors describe the use of L-carnitine at a dose of 40 mg/kg/day (2.8 g/day), and the number of deaths in the control group was one as against none in the treated group. Moreover, in this study, the treated group was divided into two subgroups, one of which was treated with L-carnitine within 4 hours of the onset of infarct symptoms, while the other was treated more than 4 hours after the onset of infarct symptoms. In their discussion of the results, the authors state that they found no significant difference between patients treated within 4 hours of the onset of infarct symptoms and those treated more than 4 hours after the onset of such symptoms.

In another publication entitled “Clinical aspects of human carnitine deficiency” published by Pergamon Press in 1986, the authors describe a blind clinical trial in which 351 patients with acute myocardial infarction were recruited, whose infarct symptoms had started within 8 hours of the start of treatment with L-carnitine. In this clinical trial, the patients received 3 grams of L-carnitine every 8 hours (9 grams a day) by the intravenous route, and the L-carnitine treatment was continued for 48 hours (the control group received saline solution). The mortality analysis revealed that there was no significant difference between the control group and the L-camitine-treated group at 7 days after the start of treatment.

This provides further confirmation of the fact that the known technique not only does not demonstrate or suggest the use of L-carnitine in the early stages after onset of an infarction in order to reduce the number of deaths, but, if anything, prejudices one technically against such use in that L-camitine has the same effect whether used within the first few hours of an infarction or later.

In the medical field it is very important to use drugs at the time most suitable for treating a given disease, such as, for example, acute myocardial infarction. The above-mentioned beta-blockers prove significantly more active if used in the early stages of onset of an infarction.

A given number of patients with acute myocardial infarction continue to die in the first week of hospitalisation and later, even when treated with all appropriate and available pharmacological and technical means. Furthermore, L-carnitine alone in the therapeutic regimens adopted to date and described in the above-mentioned publications, or in combination with said suitable and available pharmacological and technical means, though improving the treated patient's general condition, fails to reduce the number of deaths as compared to patients treated with the normal drugs used.

There is therefore a strongly perceived need for the availability of new and known drugs which are useful for reducing the number of deaths due to acute myocardial infarction, where said drugs are used alone or in combination with the normal known drugs which alone would not be capable of saving from death that proportion of patients who die all the same within the first week or later after the onset of infarction.

Thus, the aim of the present invention is to improve survival of subjects with acute myocardial infarction within the early stages of the disease, such as from the first week up to 3 months.

It has now been found, surprisingly and unexpectedly, that L-carnitine or one of its pharmaceutically acceptable salts is capable of reducing the number of deaths caused by acute myocardial infarction, and of improving the prognosis in the short and long term in the patients treated with it, in which said L-carnitine is administered intravenously within the first few hours of onset of AMI symptoms at an initial dose of 9 grams a day for 5 days, after which the treatment is continued at a dose of 4 grams a day by mouth.

What is meant by a pharmaceutically acceptable salt of L-carnitine is any salt of the latter with an acid that does not give rise to toxic or side effects.

These acids are well known to pharmacologists and to experts in pharmacy. Examples of such salts, though not exclusively these, are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethane sulphonate, magnesium 2-amino-ethane sulphonate, methane sulphonate, choline tartrate, trichloroacetate, and trifluoroacetate.

What is meant by a pharmaceutically acceptable salt of L-carnitine, moreover, is an FDA-approved salt listed in Int. J Pharm. 33 (1986), 201-217, which is incorporated herein for reference purposes.

As defined in Merck Manual 18 ed., myocardial infarction (MI) affects predominantly the left ventricle (LV), but damage may extend into the right ventricle (RV) or the atria. RV infarction usually results from obstruction of the right coronary or a dominant left circumflex artery; it is characterized by high RV filling pressure, often with severe tricuspid regurgitation and reduced cardiac output. An inferoposterior infarction causes some degree of RV dysfunction in about half of patients and produces hemodynamic abnormality in 10 to 15%. RV dysfunction should be considered in any patient who has inferoposterior infarction and elevated jugular venous pressure with hypotension or shock. RV infarction complicating LV infarction may significantly increase mortality risk. Anterior infarcts tend to be larger and result in a worse prognosis than inferoposterior infarcts. They are usually due to left coronary artery obstruction, especially in the anterior descending artery; inferoposterior infarcts reflect right coronary or dominant left circumflex artery obstruction. Transmural infarcts involve the whole thickness of myocardium from epicardium to endocardium and are usually characterized by abnormal Q waves on ECG. Nontransmural or subendocardial infarcts do not extend through the ventricular wall and cause only ST-segment and T-wave (ST-T) abnormalities. Subendocardial infarcts usually involve the inner ⅓ of myocardium, where wall tension is highest and myocardial blood flow is most vulnerable to circulatory changes. These infarcts may follow prolonged hypotension. Because the transmural depth of necrosis cannot be precisely determined clinically, infarcts are usually classified by the presence or absence of ST-segment elevation or Q waves on the ECG. Volume of myocardium destroyed can be roughly estimated by the extent and duration of CK elevation.

The present invention deals with acute myocardial infarction in general, and it is not limited to a specific location in the heart, therefore the acute myocardial infarction is not localized within a specific region of the organ but it is located anywhere in the heart.

One object of the present invention therefore is the use of L-carnitine or one of its pharmaceutically acceptable salts for the preparation of a medicine useful for reducing the number of deaths caused by acute myocardial infarction and for improving the short- and long-term prognosis in the patients treated, in which L-carnitine is administered intravenously within the first few hours of onset of the symptoms of acute myocardial infarction at an initial dose of 9 grams a day for 5 days, after which the treatment is continued at a dose of 4 grams a day by mouth.

A further object of the present invention is the use of L-carnitine or one of its pharmaceutically acceptable salts for the preparation of a medicine useful for reducing the number of deaths caused by acute myocardial infarction and for improving the short- and long-term prognosis in the patients treated, in which L-carnitine is administered intravenously within 6 hours of onset of the symptoms of acute myocardial infarction at an initial dose of 9 grams a day for 5 days, after which the treatment is continued at a dose of 4 grams a day by mouth.

A further object of the present invention is the use of L-carnitine or one of its pharmaceutically acceptable salts for the preparation of a medicine useful for reducing the number of deaths caused by acute myocardial infarction and for improving the short- and long-term prognosis in the patients treated, in which L-carnitine is administered intravenously within 4 hours of onset of the symptoms of acute myocardial infarction at an initial dose of 9 grams a day for 5 days, after which the treatment is continued at a dose of 4 grams a day by mouth.

A further object of the present invention is the use of L-carnitine or one of its pharmaceutically acceptable salts in combination with one or more known drugs, and/or known mechanical and/or surgical techniques, which alone would fail to reduce the number of deaths in infarct victims, for the preparation of a medicine useful for reducing the number of deaths caused by acute myocardial infarction and for improving the short- and long-term prognosis in the patients treated with it, in which L-carnitine is administered intravenously within the first few hours of onset of the symptoms of acute myocardial infarction at an initial dose of 9 grams a day for 5 days, after which the treatment is continued at a dose of 4 grams a day by mouth.

A further object of the present invention is the use of L-carnitine or one of its pharmaceutically acceptable salts in combination with one or more known drugs, and/or known mechanical and/or surgical techniques, which alone would fail to reduce the number of deaths in infarct victims, for the preparation of a medicine useful for reducing the number of deaths caused by acute myocardial infarction and for improving the short- and long-term prognosis in the patients treated, in which L-carnitine is administered intravenously within 6 hours of onset of the symptoms of acute myocardial infarction at an initial dose of 9 grams a day for 5 days, after which the treatment is continued at a dose of 4 grams a day by mouth.

A further object of the present invention is the use of L-carnitine or one of its pharmaceutically acceptable salts in combination with one or more known drugs, and/or known mechanical and/or surgical techniques, which alone would fail to reduce the number of deaths in infarct victims, for the preparation of a medicine useful for reducing the number of deaths caused by acute myocardial infarction and for improving the short- and long-term prognosis in the patients treated, in which L-carnitine is administered intravenously within 4 hours of onset of the symptoms of acute myocardial infarction at an initial dose of 9 grams a day for 5 days, after which the treatment is continued at a dose of 4 grams a day by mouth.

Examples of said known drugs used in intensive care which alone would fail to reduce the number of deaths in infarct victims are, though not exclusively, the following: beta-blockers, calcium antagonists, aspirin, angiotensin converting enzyme inhibitors, or ACE inhibitors, in which said ACE inhibitor is selected from the group consisting of alacepril, benazepril, benazeprilat, captopril, ceronapril, cilazapril, delapril, enalapril, enaprilat, fosinopril, imidapril, indolapril, lisinopril, moveltipril, perindopril, pentopril, pivalopril, quinapril, ramipril, spirapril, temocapril, trandolapril or zofenopril.

The preferred calcium antagonists are dilthiazem, nifedipine, verapamil, nicardipine and nimodipine.

The preferred mechanical and/or surgical techniques are angioplasty and by-pass.

The following example illustrates the invention.

### EXAMPLE 1

A clinical trial was conducted in order to evaluate the effect of the administration of L-carnitine on the incidence of mortality and heart failure in the short and long term in patients with acute myocardial infarction. The acute myocardial infarction has not localized within a specific region of the organ but it could be located anywhere in the heart. The trial design was that of a multicentre, parallel-group, double-blind, placebo-controlled, randomised trial.

A total of 2,296 male and female patients aged below 80 years were recruited. The study compound, L-carnitine, was administered at a dose of 9 g/day i.v. for the first 5 days and 4 g/day by mouth from day 6 to day 180.

Concomitant therapies were given according to the procedures adopted in local clinical practice.

The efficacy endpoints of the trial consisted in the reduction of mortality and heart failure.

### Inclusion Criteria

Typical chest pain lasting>30 minutes, not resolved by the oral or intravenous administration of nitrates;
ECG with ST segment deviation≧0.2 mV in D, and aVL and/or in two or more contiguous precordial leads;
Time interval elapsing from onset of symptoms to trial randomisation<12 hours;
Age<80 years;
Written informed consent.

### Exclusion Criteria

Pregnancy or breast-feeding;
Haemodynamically significant valvulopathy;
Hypertrophic or dilated cardiomyopathy;
Congenital heart disease;
Clinically severe liver or kidney disease;
Alcohol abuse;
Other diseases associated with a poor life expectancy;
Conditions making poor compliance with treatment and/or periodic visits likely;
Inclusion in another trial.
   The results obtained are presented in Table 1.

TABLE 1

| | NUMBER OF DEATHS AT: | | | | | | |
|---|---|---|---|---|---|---|---|
| | 3 | 5 | 7 | 1 | 2 | 6 | 12 |
| | days | days | days | mos | mos | mos | mos |
| Placebo | 34 | 43 | 45 | 58 | 65 | 74 | 75 |
| L-carnitine | 23 | 27 | 31 | 45 | 53 | 64 | 67 |
| RR | 0.68 | 0.63 | 0.69 | 0.78 | 0.81 | 0.86 | 0.89 |
| P | 0.1357 | 0.0498 | 0.097 | 0.1766 | 0.238 | 0.3546 | 0.4555 |

RR = Relative Risk

These results show that the compound according to the invention, with the particular treatment regimen adopted in this clinical trial, induced a statistically significant reduction in deaths after 5 days' treatment (P<0.05) and significant reductions at the other observation times.

The L-carnitine doses used according to the present invention and the treatment regimen may be varied at the discretion of the primary care physician on the basis of his or her experience and the patient's general condition, also thanks to the lack of toxicity of the compound according to the invention.

The formulations for intravenous administration, according to the present invention, consist of solutions or suspensions in suitable vehicles such as saline solution, distilled water, glucose solution, or others.

The formulations for oral administration, according to the present invention, consist of tablets, capsules, powders, granules, syrups, elixirs, solutions or suspensions.

The compound according to the invention can be administered in single or multiple doses.

When the compound according to the invention (in single or multiple doses) is administered in combination with one or more of the above-mentioned known drugs used in the intensive care which alone would fail to reduce the number of deaths in infarct victims, said combination can be administered as a single pharmaceutical composition combining the active ingredients in a pharmaceutically acceptable vehicle, or said active ingredients can be administered separately, simultaneously, or in sequence, via the same or different administration routes.

When the compound according to the present invention is administered in combination with other drugs, the administration can be effected in any suitable dosage form combination, e.g. in the form of oral L-carnitine/oral drug used in combination with it; or injectable L-carnitine/oral drug used in combination with it; or oral L-carnitine/injectable drug used in combination with it.

The present invention also relates to a kit combining the active ingredients, separately, in a single pack.

This kit is particularly useful when the components have to be administered by different routes and/or at different times.

1. A method of reducing the number of deaths caused by acute myocardial infarction located anywhere in the heart within the first week comprising intravenously administering L-carnitine or one of its pharmaceutically acceptable salts within the first four hours of onset of the symptoms of acute myocardial infarction at an initial dose of 9 grams a day for 5 days, after which the treatment is continued as a dose of 4 grams a day by mouth from day 6 to day 180.

2. The use according to claim 1 in which the pharmaceutically acceptable salt of L-carnitine is selected from the group consisting of chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethane sulphonate, magnesium 2-amino-ethane sulphonate, methane sulphonate, choline tartrate, trichloroacetate, and trifluoroacetate.

3. The method according to claim 1, in which the L-carnitine for oral administration is in the form of a tablet, capsule, powder, granule, syrup, elixir, suspension or solution.

4. The method according to claim 1, in which the L-carnitine for intravenous administration is in the form of a suspension or a solution in a suitable vehicle.

5. The method according to claim 4, in which the vehicle is selected from the group consisting of distilled water, a saline solution and a glucose solution.
